(19) United States
(12) Patent Application Publication
(10) Pub. No.: US 2008/0227210 A1
(43) Pub. Date: Sep. 18, 2008

(54) Home test for glycated albumin in saliva

(76) Inventor: Henry Smith, Temecula, CA (US)

Correspondence Address: HENRY J. SMITH, 40670 CALLE TORCIDA, TEMECULA, CA, 92591, US

(21) Appl. No.: 12/069,165

(22) Filed: Feb. 8, 2008

Related U.S. Application Data
(60) Provisional application No. 60/906,322, filed on Mar. 12, 2007.

(51) Int. Cl. G01N 33/00; G01N 1/14; G06F 17/40

(52) U.S. Cl. 436/86; 73/864; 702/187

(57) ABSTRACT

A home test for measuring glycated albumin levels in saliva. The saliva sample is collected at home using a standardized saliva collection kit and mailed to a testing laboratory that performs the test and reports the result directly back to the customer via the internet. The home test can be used to monitor glucose control in diabetics and in healthy individuals. It may also be used as a diagnostic aid in identifying individuals with diabetes, or who are at risk of developing diabetes.

### CROSS-REFERENCE TO RELATED APPLICATIONS

This utility patent application claims priority to Provisional Patent Application Ser. No. 60/906,322 filed Mar. 12, 2007 entitled HOME TEST FOR GLYCATED ALBUMIN IN SALIVA.
World Health Organization. Diabetes Statistics 2006.
LifeScan, Inc. Product Brochure One Touch Brand Glucose Meter. Product Insert.
Flexsite Diagnostics, Inc. Website information www.flexsite.com
Metrika, Inc. A1C Now. Product Insert.
Exocell, Inc. Glycaben. Product Insert.
Smith H. J. and Zaidi, A. Rapid Test for Glycated Albumin.(WO 2005/031356 A1).

### FEDERALLY SPONSORED RESEARCH OR DEVELOPMENT

None

### BACKGROUND OF THE INVENTION

There are 20.8 million diabetics in the US. About 14.6 million are confirmed diabetics; and it is estimated that there are another 6.2 million that are undiagnosed diabetics. In addition it is estimated that there are a further 54 million who are prediabetic or are at risk of developing diabetes. (American Diabetes Association). The incidence of diabetes is estimated to be 180 million worldwide. (World Health Organization data).

Diabetics have elevated levels of glucose in their blood. Excessive levels of blood glucose bind to proteins causing them to become glycated. Glycated proteins cannot function normally and excessive levels of glycated proteins will result in tissue damage. The process of progressive tissue damage can occur over many years without overt clinical signs. However, if the disease progresses far enough clinical signs will eventually appear and depending on the tissue most affected may present as blindness; or kidney failure; or ulcers that will not heal properly.

There are two types of diabetes. Type 1 diabetics lack functional insulin and must take daily insulin to maintain good glucose control. They make up approximately 5% of the diabetic population. The other 95% of diabetics are Type 2 diabetics where their insulin levels appear to be normal but there is failure to metabolize glucose properly resulting in elevated levels of blood glucose.

There are a variety of tests available to monitor blood glucose levels and to assess the level of glycated proteins.

The blood glucose test is available as a home test consisting of a glucose meter and disposable test strips. (e.g. One Touch Brand Glucose Meter). It is primarily used by Type 1 diabetics to monitor their blood glucose levels throughout the day so that they can inject the correct amount of insulin to prevent excessive glucose levels from occurring. Type 2 diabetics do not take insulin and therefore do not need to perform daily blood glucose testing.

The blood glucose test provides a point-in-time result and does not provide an overall assessment of the average level of blood glucose over time. To monitor average blood glucose levels over time and to obtain a more direct assessment of tissue damage several tests were developed to measure the level of glycated proteins in the blood.

The hemoglobin A1c (or glycated hemoglobin) test measures the amount of glycated hemoglobin compared to total hemoglobin in a blood sample. It provides an overall assessment of glucose control over the preceding 2-3 months. It is generally performed in a reference laboratory. To facilitate convenience for the patient a mail-in home test was developed where the patient mails a blood sample to a reference laboratory which then mails back the test result (Flexsite Diagnostics, Inc.). Also, Metrika, Inc. has announced the development of a fully self-contained home and point-of-care hemoglobin A1c test that utilizes test strips and a measuring instrument.

In order to gain an overall assessment of glucose control over a shorter time frame of the preceding 2-3 weeks several tests for measuring glycated albumin in blood have been developed. Exocell, Inc. is marketing a test kit for measuring glycated albumin in blood that is performed in reference laboratories; and Smith H. J. and Zaidi A. describe a fully self-contained home glycated albumin test that utilizes test strips and a measuring instrument (WO 2005/031356 A1).

All the current tests for monitoring glucose control involve collecting a blood sample for analysis. It would be desirable to develop a test system that can utilize a non-invasive sample such as saliva for analysis. It would also be desirable to develop a test that is convenient and easy to use.

This invention describes the development of a home test for glycated albumin in saliva that involves the collection of the saliva sample; mailing the sample to a testing laboratory where it is tested; and retrieving the result from an electronic data base via the internet.

### BRIEF SUMMARY OF THE INVENTION

The level of glycated albumin in saliva provides an assessment of glucose control in the individual. The individual collects a sample of saliva and mails the specimen to a reference laboratory which performs the analysis. The laboratory result is stored numerically and graphically in a data-base maintained by the reference laboratory. Sequential laboratory test results are maintained for each individual so that trend analysis can be performed. The individual can access his or her personal laboratory result via the internet by accessing the laboratory's web-site and entering the appropriate kit identifier code into a computer. In addition to the report there is also information presented to assist the patient in interpreting the result; and links to other sites that can be of use to those seeking further assistance.

### BRIEF DESCRIPTION OF THE DRAWINGS

FIG. 1. is a flow diagram of the overall process.

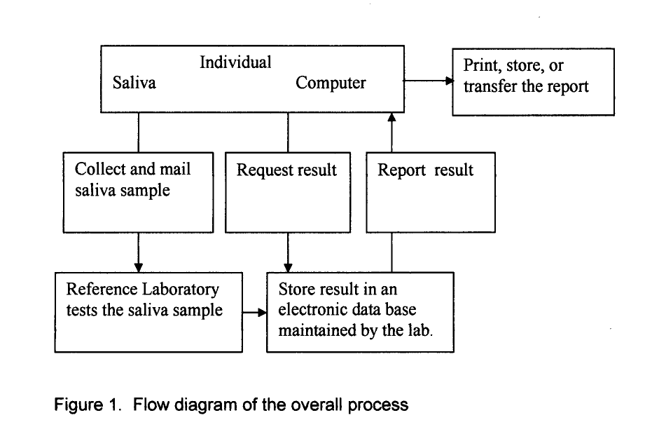

FIG. 2. is an example of a test report. The actual values of the reference range may change depending on factors such as age, gender, race etc.

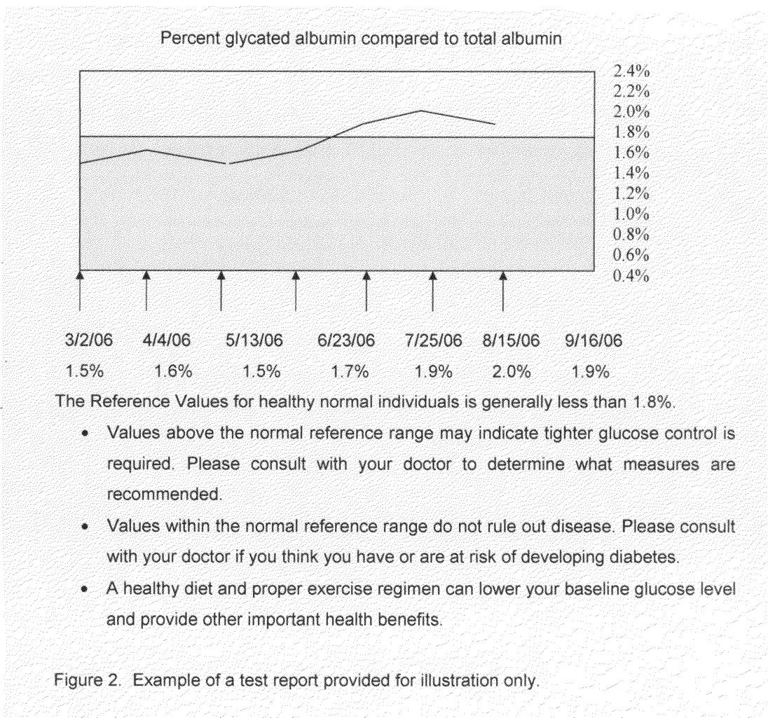

### DESCRIPTION OF THE INVENTION

This invention is novel in that there is no prior art describing a mail-in home test for glycated albumin in saliva. This invention describes a home test for glycated albumin in saliva that consists of the following steps: a saliva sample is collected and mailed to a reference laboratory which tests the sample and reports the glycated albumin result directly back to the individual via the internet.

To use the test the individual has to follow the saliva sample collection and mailing instructions provided with the kit. A test kit may be in a single test configuration containing the following components: an exterior box or container enclosing one bar-coded or radio-frequency identification (RFID) tagged saliva collection tube with leak-proof cap; one straw; one leak-proof resealable plastic pouch; one absorbent pad; and one preaddressed pre-paid mailing container. The test kit may also be provided in a multiple test mailing format to allow sequential testing and monitoring of glycated albumin level with time. In the multiple test kit pack all the contents within the exterior container will be multiplied by the number of tests to be performed. For example, a six test kit pack will consist of an exterior container enclosing six bar-coded or RFID tagged saliva collection tubes with leak-proof caps; six straws; six leak-proof plastic resealable plastic pouches; six absorbent pads; and six preaddressed pre-paid mailing containers. Other multiple test kit packs can be similarly configured. Each test kit pack will have its own unique barcode or RFID tag for that kit. Each kit will also contain a product insert with instructions on how to collect the saliva specimen; mailing instructions; instructions on how to access the test result using the internet; and assistance in how the result should be interpreted.

Specimen Collection.

Before the sample is collected the mouth should be rinsed with clean water. Saliva is allowed to collect in the mouth and then dribbled thru the straw into the leak-proof specimen collection tube. About 1 ml of saliva is required although the test can be performed on much less. The specimen collection tube is then capped and placed in a leak-proof plastic pouch along with an absorbent pad (e.g. blotting paper or similar material) to absorb any leakage. The pouch is then placed in the pre-addressed, pre-paid mailing container and mailed to the testing laboratory. The mailing container and mailing instructions are in accordance with US postal regulations. Although the saliva sample can be collected at any time it is recommended that collection be performed early in the day so that the specimen can be mailed the same day.

Laboratory Testing.

Upon arrival in the laboratory the labeled specimen is scanned with a bar code or RFID scanner and the data is used to prepare an electronic file on that particular coded specimen in the data base. The data entered is also used to prepare a test worksheet. To measure glycated albumin in the sample two different assays are employed. One assay is used to measure the level of glycated albumin in the sample compared to a standard curve prepared from a set of known glycated albumin standards, and another assay is used to measure the total albumin in the sample compared to a standard curve prepared from a set of known albumin standards. The two results are then used to calculate the percentage of glycated albumin compared to total albumin in the sample. The result and the sample collection date are saved in the electronic data file that is unique to that sample's identifier code. Successive test samples received over time from the same test kit are tested and entered into the unique data file for that kit. The results are stored numerically and graphically so that the level of glycated albumin can be monitored over time.

Test Results.

Persons who have submitted saliva samples for testing can access their laboratory test report by logging in to the testing laboratory's website. There they will enter the alphanumeric sequence of the identifier code for their kit in a specific field that requests this information. If this is the first time that the kit's identifier code is entered a new file is prepared in the testing laboratory's electronic database. The next time this information is entered the laboratory computer will access the corresponding file and will update the report and display it a numerical and graphical format. The report will also include information to assist in interpretation of the test results. The report can be printed, or stored electronically, or transmitted to another computer. The individual can only access their own security encrypted file report; and the report cannot be edited or altered in any manner by the end-user.

Test Interpretation.

There are no published studies on the level of glycated albumin in saliva in normal individuals and in diabetics. I have developed a sensitive laboratory assay for measuring the low levels of glycated albumin present in saliva. I have also developed a sensitive laboratory assay for measuring the low level of albumin present in saliva. By measuring the level of glycated albumin and total albumin from the same saliva sample it is possible to calculate the amount of glycated albumin compared to total albumin in the sample. The result is expressed as the percentage of glycated compared to total albumin in the sample. Clinical studies were conducted to develop a “normal” reference range for glycated albumin in saliva for normal healthy persons. In contrast, many diabetics with poor glucose control have elevated levels of glycated albumin in their saliva. The test can be used 1) as a diagnostic test to aid in diagnosing individuals who have diabetes or who are pre-diabetic or who are at risk of developing diabetes and/or 2) to monitor the individual's glucose control over time so that appropriate preventative and therapeutic measures can be implemented in a timely fashion.

Glycated Albumin Test for Monitoring Glucose Control Over Time.

In order to monitor the average level of glycated albumin with time it is recommended that the test be performed on a monthly schedule. This is because saliva albumin is made up of albumin from blood that has migrated thru the salivary glands and secreted as saliva. Albumin in blood has a circulating life of about 20 days and therefore the level of glycated albumin in blood represents the average glycated albumin level over approximately the preceding two weeks. As saliva albumin is derived from blood it will also provide an estimate of the average glycated albumin level over the preceding two weeks.

Discussion

The current tests for diagnosing diabetes are the Fasting Plasma Glucose Test and/or the Oral Glucose Tolerance Test which are done under medical supervision in a doctor's office.

The home glucose test provides a point-in-time result that is important for diabetics who need to adjust their daily insulin dosage. The glucose test however cannot provide an overall assessment of glucose control over time. While the hemoglobin A1c test does provide an overall assessment of glucose control over an extended period of time of several months it does not allow for an early therapeutic response. The hemoglobin A1c test requires a blood sample which is sent to a testing laboratory. The glycated albumin in blood test does provide an overall assessment of glucose control over a shorter time frame of several weeks, but currently the patient has to have a blood sample taken and the test sent to a reference laboratory for analysis.

This invention describes several significant advantages over earlier tests. The main advantage being that the test is performed on a saliva sample. The advantage of not having to collect a blood sample, particularly in children, is significant. Having the testing performed at a reference laboratory with standardized procedures and skilled staff is also advantageous as all the errors associated with having an untrained individual do the testing at home are eliminated. The method of reporting results using the internet is also an improvement over the current method of mailing or faxing the result back to the client.

This invention describes a streamlined home test for measuring glycated albumin in saliva. The development of an accurate, timely, convenient and non-invasive test that can be ordered from home and reported directly back to the client will have important implications for diabetics and those at risk of developing diabetes.

In one embodiment of this invention the glycated albumin in saliva test is used to monitor glucose control in diabetics; prediabetics and in those individuals believed to be at risk of developing diabetes. In may also be used by healthy individuals who wish to monitor their health status.

In another embodiment of this invention the glycated albumin in saliva test is used as a screening test in conjunction with other laboratory and clinical signs to diagnose diabetes and/or to identify individuals at risk of developing diabetes.

The above description is given by way of example, and not limitation. For example, the inventor has also developed a portable device for measuring glycated albumin in saliva that can be used at home and/or in doctor's offices and/or in medical laboratories. Said device can also be used by the testing laboratory to measure glycated albumin levels in saliva samples that are mailed to the testing laboratory; and the laboratory test results accessed by the individual in like manner as described in this invention. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

1. A home test for measuring glycated albumin level in saliva consisting of the following steps:
a method of saliva collection
a method of sample identification
a method of mailing the sample to a reference laboratory
a laboratory testing process to obtain a result
a report of the test result and guidance on interpretation of the result
a method of reporting the result via the internet.

2. A method of saliva collection according to claim 1 using a drinking straw to allow the saliva to flow into a leak-proof collection tube or container.

3. A method of mailing the sample to a testing laboratory according to claim 1 using a pre-addressed and pre-paid mailing container.

4. A method of identifying each person's sample according to claim 1 using a identifier code unique to each kit.

5. A laboratory testing process according to claim 1 to obtain a single test result, or successive test results obtained on successive samples tested periodically.

6. A method of calculating the result and presenting successive test results in a numerical and graphical format according to claim 1 so that trend analysis can be done.

7. An electronically stored laboratory report according to claim 1 that is encrypted and only accessible to the end-user via the internet by entering the identifier code sequence.

8. A method according to claim 1 of monitoring glucose control over a period of time by performing periodic measurements of glycated albumin in saliva using the home test for glycated albumin in saliva.

9. A method according to claim 1 of diagnosing diabetes using the home test for glycated albumin in saliva.

10. A method according to claim 1 of diagnosing pre-diabetes using the home test for glycated albumin in saliva.

11. A method according to claim 1 for diagnosing individuals that may be at risk of developing diabetes using the home test for glycated albumin in saliva.

12. A method according to claim 1 for healthy individuals to monitor their general health status using the home test for glycated albumin in saliva.
